(19) United States
(12) Patent Application Publication
(10) Pub. No.: US 2025/0041233 A1
(43) Pub. Date: Feb. 6, 2025

(54) FORMULATIONS OF ULTRAVIOLET (UV) DEGRADABLE MATERIALS AND USES THEREOF

(71) Applicant: The Johns Hopkins University, Baltimore, MD (US)

(72) Inventors: Christopher M. Hoffman, JR., Odenton, MD (US); Scott A. Shuler, Baltimore, MD (US); Lauren K. Ratliff, Baltimore, MD (US); Zachary H. Kiick, Ellicott City, MD (US)

(73) Assignee: The Johns Hopkins University, Baltimore, MD (US)

(21) Appl. No.: 18/791,927

(22) Filed: Aug. 1, 2024

Related U.S. Application Data
(60) Provisional application No. 63/517,358, filed on Aug. 3, 2023.

(51) Int. Cl. A61K 9/50; A61K 45/06

(52) U.S. Cl. CPC A61K 9/5031; A61K 45/06

(57) ABSTRACT

A microcapsule includes an oxime-based polymerization product of two or more monomers and a payload. The payload may be encapsulated within the microcapsule. The oxime-based polymerization products may include an oxime-based monomer and an isocyanate-based monomer. Methods of making such microcapsules and methods of delivering a payload to a target using such microcapsules are provided.

### CROSS-REFERENCE TO RELATED APPLICATIONS

This application is related to U.S. Provisional Application No. 63/517,358, filed Aug. 3, 2023, the contents of which are incorporated by reference herein in their entirety.

### BACKGROUND OF THE INVENTION

Microencapsulation is a physicochemical or mechanical process whereby a material of interest or a payload may be embedded in another material, typically a polymer, to form microcapsules having a diameter from a few nanometers to about a centimeter. Microcapsules may be used, for example, to (i) provide protection and stability to payloads entrapped inside of the microcapsule, (ii) provide a controlled release of the payload, (iii) extend the lifetime of a payload in an environment of interest, (iv) reduce the threat of exposures to the payload, and/or (v) provide a method of easily handling the entrapped payload which may otherwise be toxic or difficult to handle. Microcapsules that release a payload in response to specific stimuli may be of interest in a multitude of industries including, for example, the pharmaceutical, agriculture, food, textile, and/or cosmetic industries because of the aforementioned benefits provided.

### SUMMARY OF THE INVENTION

According to some embodiments, a microcapsule may comprise an oxime-based polymerization product of two or more monomers, wherein a payload is encapsulated within the microcapsule.

According to some embodiments, for a method of preparing a plurality of microcapsules, the method may comprise: preparing a solution A comprising a nanoclay and mineral oil; preparing a solution B comprising a payload and an oxime-based monomer; preparing a solution C comprising an isocyanate-based monomer and mineral oil; adding a polymeric amine to solution B; adding solution B to solution A to obtain a solution D; adding solution C to the solution D to obtain a solution E; covering solution E to prevent exposure to light; and at least one of mixing, shaking, or stirring the covered solution E at room temperature for a predetermined time to obtain the plurality of microcapsules dispersed in solution, the microcapsules encapsulating portions of the payload.

According to some embodiments, for a method of delivering a payload to a target, the method may comprise: applying microcapsules to the target, the microcapsules encapsulating the payload, the microcapsules comprising an oxime-based polymerization product of two or more monomers; and exposing the target to ultraviolet light to decompose the microcapsule and release the payload to the target.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows example microcapsules of the disclosure at a macroscopic, microscopic, and SEM views before and after exposure to UV light.

FIG. 2 shows SEM images of example microcapsules of the disclosure before and after exposure to UV light for 30 minutes.

FIG. 3 shows SEM images of example microcapsules of the disclosure degrading over various exposure times in both a UV oven and sunlight.

FIG. 4A shows a SEM image of an example microcapsule of the disclosure after 10 minutes of UV exposure.

FIG. 4B shows a SEM image of an example microcapsule of the disclosure after 10 minutes of heat exposure.

FIG. 5 shows an example method for preparing an example microcapsule of the disclosure.

FIG. 6 shows an example method for extracting an example microcapsule of the disclosure.

FIGS. 7A-7C show a generic scheme of a payload being encapsulated in example microcapsules of the disclosure and released upon exposure to UV resulting in the interactions of the payload and a target substrate.

FIG. 8 shows fluorimetry data from a control study of a cellulase enzyme.

FIG. 9 shows fluorimetry data from a control study of a cellobioside-resorufin substrate.

FIG. 10 shows results from a comparative study between example microcapsules.

FIG. 11 shows results from a comparative study between example microcapsules that were exposed to heat versus example microcapsules that were exposed to UV light.

### DETAILED DESCRIPTION OF THE EMBODIMENTS

As recognized by the inventors, microcapsules may be designed to respond to stimuli of specific chemical conditions (e.g., acidic or basic conditions, temperature, magnetic field, or electric field). However, few examples have been demonstrated to date.

UV degradable materials have traditionally utilized a compound bearing a nitro or NO₂ substituent to provide an adequate chromophore and facilitate light-induced degradation. However, as recognized by the inventors, nitro-containing compounds are suboptimal reagents for the preparation of UV degradable materials due to, for example: their toxicity, their propensity to form energetic intermediates; and the highly colored nature of nitro-containing compounds. Moreover, as recognized by the inventors, nitro-containing compounds may degrade readily when exposed to light, thus providing little control over the degradation rate of the material.

As recognized by the inventors, novel UV degradable materials are required to overcome these challenges. Further, as recognized by the inventors, there is a need for the development of novel UV degradable materials that can be used to prepare UV degradable microcapsules that may offer, for example: improved stability of the microcapsule; improved control over the release of a payload; and/or eliminate the need for toxic and energetic nitro-containing compounds. Moreover, as recognized by the inventors, there is a need for new methods to prepare UV degradable microcapsules that are tolerant of a variety of payloads.

As discovered by the inventors, polymers comprising an oxime-based monomer may afford a polymer that is UV degradable and may offer improved properties when applied to UV degradable microcapsules. As discovered by the inventors, microcapsules comprising an oxime-based polymerization product may afford a microcapsule that can deliver a payload (e.g., an enzyme), may offer improved stability of the microcapsule, and may provide a tunable release of the payload. This novel and unobvious invention may overcome the present limitations in UV degradable microcapsules, while also mitigating the risks associated with the nitro-containing polymers that may be used in the preparation of UV degradable microcapsules.

In some embodiments, a microcapsule may comprise an oxime-based polymerization product of two or more monomers and a payload. In some embodiments, the oxime-based polymerization product may be comprised of an oxime-based monomer and an isocyanate-based monomer. In some embodiments, the payload may be encapsulated within the microcapsule.

In some embodiments, the oxime-based monomer may contain one or more oxime functional groups. In some embodiments, the oxime-based monomer may contain one, two, or three oxime functional groups. In some embodiments, the oxime-based monomer may be one of the general formulae

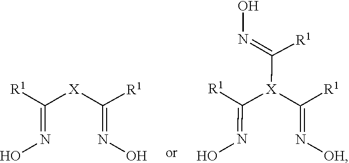

wherein X is a linking moiety. In some embodiments, the linking moiety X, may comprise at least 1 and at most 30 carbon atoms. In some embodiments, the linking moiety X may be an C₁₋₃₀alkyl, C₁₋₃₀cycloalkyl, C₁₋₃₀aryl, or C₁₋₃₀heteroaryl group. As will be understood by one of ordinary skill in the art, the oxime-based monomer referenced herein may be derived from any compound containing one or more carbonyl functional groups. According to some embodiments, a compound containing one or more carbonyl functional groups, when referred to as the oxime-based monomer, may be reacted to form an oxime functional group at one or more of the carbonyl functional groups. In some embodiments, the oxime-based monomer may be selected from the group consisting of acetovanillone, progesterone, and 1,3,5-triacetylbenzene. In some embodiments, the oxime-based monomer may be selected from the group consisting of:

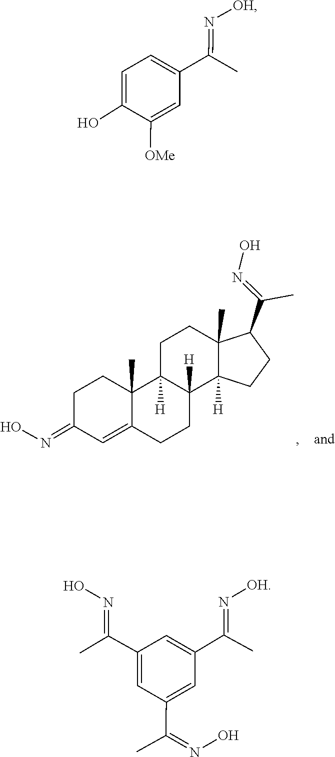

In some embodiments, the isocyanate-based monomer may contain two or more isocyanate functional groups. In some embodiments, the isocyanate-based monomer may contain two or three isocyanate functional groups. In some embodiments, the isocyanate-based monomer may be of the general formulae

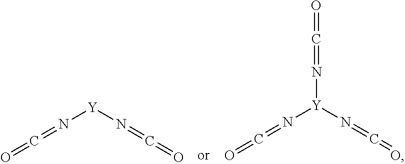

wherein Y is a linking moiety. In some embodiments, the linking moiety Y may comprise at least 1 and at most 30 carbon atoms. In some embodiments, the linking moiety Y may be an C₁₋₃₀alkyl, C₁₋₃₀cycloalkyl, C₁₋₃₀aryl, or C₁₋₃₀heteroaryl group. In some embodiments, the isocyanate-based monomer may be selected from the group consisting of isophorone diisocyanate, methylene diisocyanate, hexamethylene diisocyanate, and toluene diisocyanate.

In some embodiments, the oxime-based polymerization product may further comprise a polymeric amine. In some embodiments, the polymeric amine may contain primary and/or secondary amine functional groups. In some embodiments, the polymeric amine may be selected from the group consisting of polyethyleneimine, poly(allylamine), polyvinylamine, poly(4-aminostyrene), and chitosan. In some embodiments, the polymeric amine may be polyethyleneimine. In some embodiments, the polyethyleneimine may have an average molecular weight between at least 500 and at most 1,000 kilodaltons.

In some embodiments, the oxime-based monomer, the isocyanate-based monomer, and the polymeric amine may be combined in different ratios. In some embodiments, the nucleophilic components (e.g., the oxime-based monomer and polymeric amine) and the isocyanate-based monomer may be combined in a 3:1 ratio, a 2:1 ratio, a 1:1 ratio, a 1:2 ratio, or a 1:3 ratio. In some embodiments, the nucleophilic components (e.g., the oxime-based monomer and polymeric amine) and the isocyanate-based monomer may be combined in about a 3:1 ratio, about a 2:1 ratio, about a 1:1 ratio, about a 1:2 ratio, or about a 1:3 ratio. In some embodiments, the nucleophilic components and the isocyanate-based monomer may be combined in about a 1:1 ratio. In some embodiments, the oxime-based monomer, the isocyanate-based monomer, and the polymeric amine may be in a 1:2:1 ratio.

In some embodiments, the microcapsules may be extracted and dried to afford the microcapsules as a solid powder. In some embodiments, the microcapsules may be dispersed in a solution. In some embodiments, the solution may be an oil. In some embodiments, the oil may be a mineral oil. In some embodiments, the oil may be a heavy mineral oil.

In some embodiments, the microcapsules may include a payload, wherein the payload may be encapsulated within the microcapsule. In some embodiments, the interior of the microcapsule may comprise the payload and an aqueous solution. In some embodiments, a payload may be any chemical moiety that exhibits hydrophilic properties. In some embodiments, the payload may be water-soluble. In some embodiments, the payload may be an enzyme, a catalyst, or a pharmaceutical. In some embodiments, the payload may be a cellulase enzyme.

For the example microencapsulation methods described herein, particle size may generally be achieved through control of the physical conditions under which the involved processes are carried out (e.g., stir rate, temperature, reaction time, rate of addition of one or more of the reagents, etc.). In some embodiments, the microcapsules may be at least 1 micron to at most 1000 microns in diameter, or at least 5 microns to at most 500 microns in diameter, or at least 10 microns to at most 100 microns in diameter.

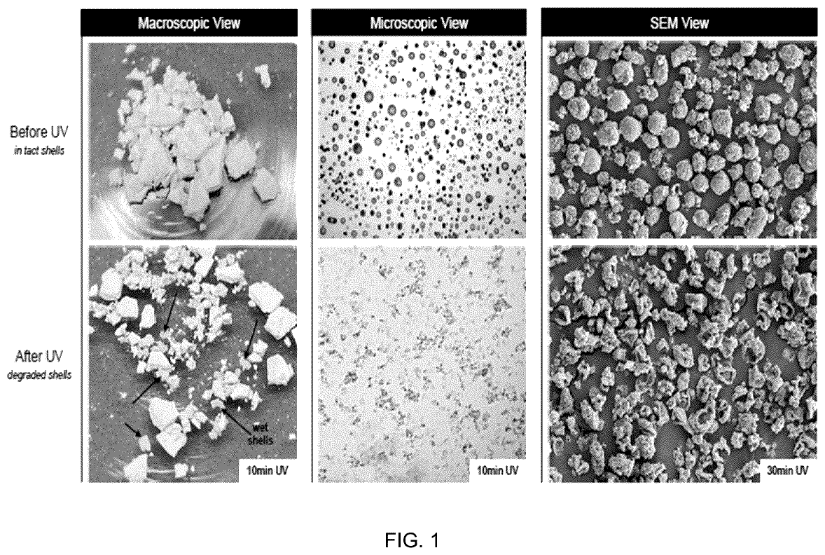

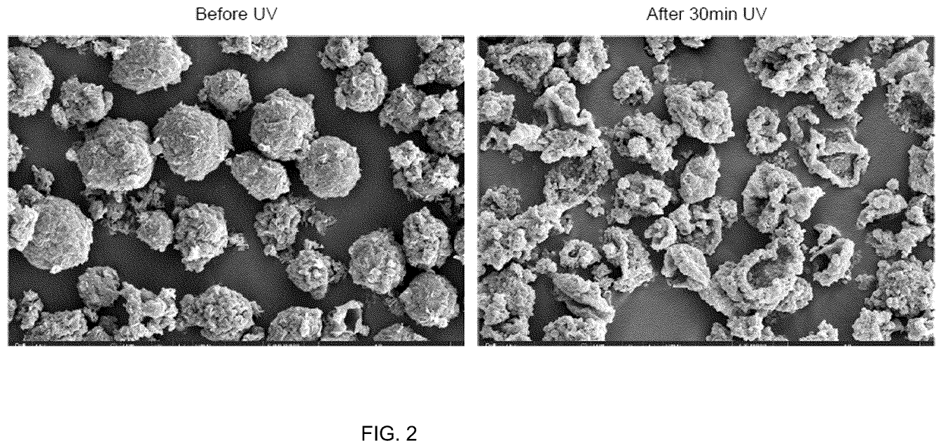

FIGS. 1-3 depict the degradation of example microcapsules of the invention when exposed to ultraviolet (UV) light. In FIG. 1, the left column shows macroscopic views and the center column shows microscopic views of example microcapsules before and after 10 minutes of exposure to UV light. In the right column of FIG. 1 and in FIG. 2, scanning electron microscopic (SEM) views show the example microcapsules before and after 30 minutes of exposure to UV light. In the microscopic and SEM views, it is clear that exposure to UV light leads to a degradation of the microcapsule that allows the release of the payload from within the microcapsule. The UV responsiveness of the microcapsules may be controlled based on the composition of the microcapsule. In some embodiments, the microcapsule may degrade when exposed to UV light. In some embodiments, the UV light source may be a UV oven or sunlight. In some embodiments, the microcapsules may degrade within minutes, hours, days, or months of exposure to UV light.

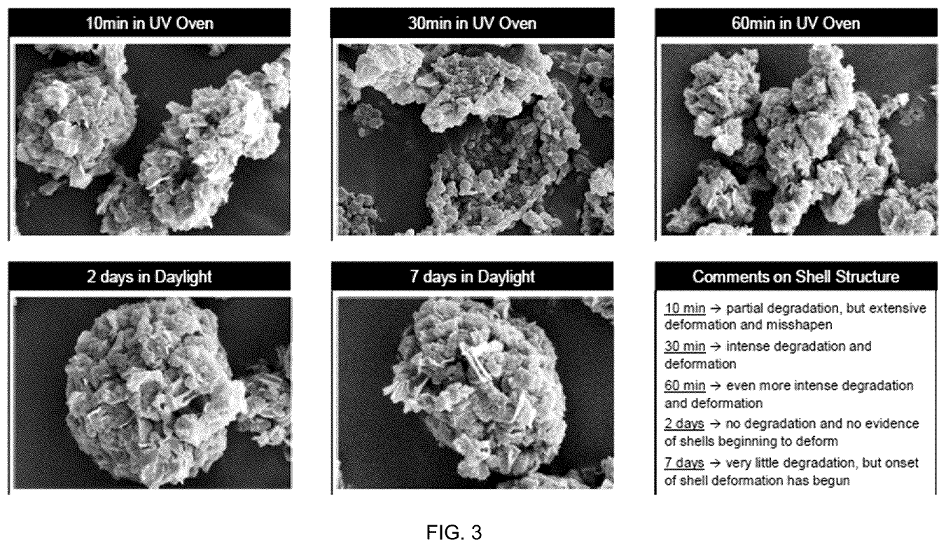

FIG. 3. depicts SEM images of example microcapsules degrading over various time periods when exposed to different sources of UV light. In the top row of FIG. 3, the microcapsules are shown to degrade in at least 10 minutes and at most 60 minutes when exposed to a UV oven. In some embodiments, the microcapsules may degrade in at least 90 minutes when exposed to a UV oven. In the bottom row of FIG. 3, the microcapsules are shown to degrade in at least 2 days and at most 7 days when exposed to sunlight (e.g., daylight). In some embodiments, the microcapsules may degrade in at least 12 hours and at most 14 days when exposed to sunlight. As shown in FIG. 3, the UV light source may provide differences in the rate of degradation of the microcapsules.

In some embodiments, different wavelengths of the light source may affect the rate of degradation of the microcapsules. In some embodiments, the microcapsule may degrade after exposure to UV light comprising wavelengths of at least 100 nm to at most 400 nm.

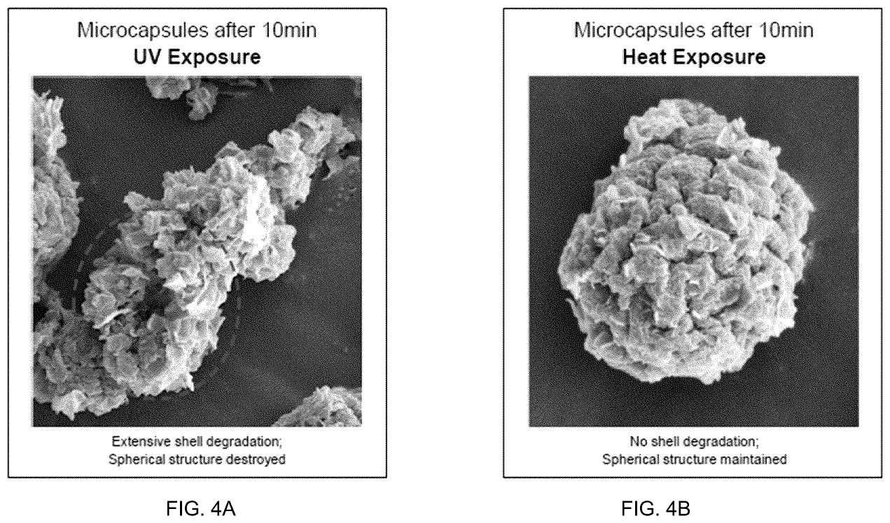

FIG. 4A-B shows SEM images of the example microcapsules after exposure to UV light and heat. Example microcapsules disclosed herein may be designed to degrade when exposed to UV light but may remain intact when exposed to other stimuli. For example, as shown in FIG. 4A, extensive degradation of the microcapsule may occur after 10 minutes of UV exposure. In contrast, as shown in FIG. 4B, the microcapsule may be resistant to heat exposure showing no degradation or deformation of the microcapsule shell after 10 minutes of heat exposure.

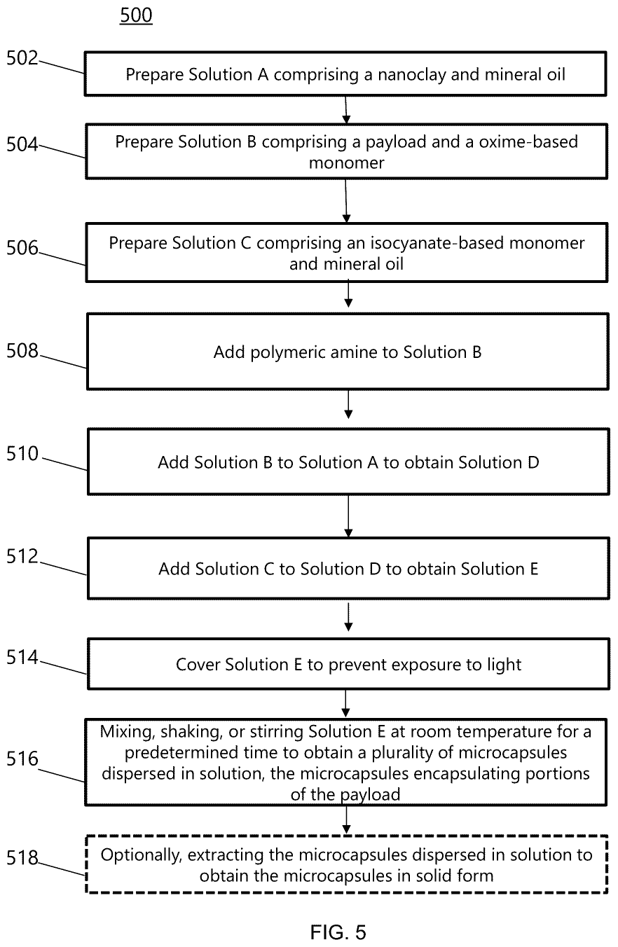

FIG. 5 depicts an example flowchart 500 for preparing example microcapsules according to one or more embodiments described herein. The flowchart 500 may be implemented for any suitable microcapsules or delivery system. While an order of operations is indicated in FIG. 5 for illustrative purposes, the timing and ordering of such operations may vary where appropriate without negating the purpose and advantages of the examples set forth in detail herein.

In step 502, a solution A may be prepared comprising a nanoclay and an oil. In some embodiments, the nanoclay may be selected from monmorillonite, kaolinite, or saponite. In some embodiments, the nanoclay may be surface modified. In some embodiments, the nanoclay may be surface modified with 35 to 45 wt % of dimethyl dialkyl (C₁₄₋₁₈) amine. In some embodiments, the oil may be a mineral oil. In some embodiments, the oil may be a heavy mineral oil. In some embodiments, solution A may be stirred between at least 5,000 rpms and at most 10,000 rpms to facilitate dissolution of or evenly disperse the nanoclay in the oil. In some embodiments, solution A may be stirred for at least 1 minutes to at most 24 hours. In some embodiments, solution A may be stirred for 30 minutes.

In step 504, a solution B may be prepared comprising a payload and an oxime-based monomer of one or more of the embodiments disclosed herein. The solvent of solution B may be a solvent suitable for dissolution of the payload and the oxime-based monomer. In some embodiments, the solvent of solution B may comprise an aqueous solution. The aqueous solution may comprise aqueous salts. In some embodiments, the solvent of solution B may comprise an aqueous buffer. In some embodiments, the solvent of solution B may further comprise a polar aprotic solvent. In some embodiments, the polar aprotic solvent may be selected from the group consisting of acetone, dimethylsulfoxide (DMSO), dimethyl formamide (DMF), dimethylacetamide (DMA), tetrahydrofuran (THF), and dichloromethane. In some embodiments, the polar aprotic solvent may be dimethylsulfoxide (DMSO). In some embodiments, DMSO may comprise at least 1% to at most 50% of the solvent of solution B. In some embodiments, solution B may be stirred between at least 5,000 rpms and at most 10,000 rpms to facilitate dissolution of or evenly disperse the payload and the oxime-based monomer throughout solution B. In some embodiments, solution B may be stirred for at least 1 minutes to at most 24 hours.

In step 506, a solution C may be prepared and may comprise an isocyanate-based monomer and an oil of one or more of the embodiments disclosed herein. In some embodiments, the oil may be a mineral oil. In some embodiments, the oil may be a heavy mineral oil. In some embodiments, solution C may be stirred between at least 5,000 rpms and at most 10,000 rpms to facilitate dissolution of or evenly disperse the isocyanate-based monomer in the oil. In some embodiments, solution C may be stirred for at least 1 minutes to at most 24 hours. In some embodiments, the viscosities of solution A, solution B, and solution C are substantially the same.

In step 508, a polymeric amine of one or more of the embodiments disclosed herein may be added to solution B. In some embodiments, the polymeric amine may be added to solution B as an aqueous solution. In some embodiments, the polymeric amine may be a polyethyleneimine. In some embodiments, solution B may be stirred between at least 5,000 rpms and at most 10,000 rpms to facilitate dissolution of or evenly disperse the oxime-based monomer, the payload, and the polymeric amine in the solution. In some embodiments, solution B may be stirred for at least 1 minute to at most 24 hours.

In step 510, solution B may be added to solution A to obtain solution D. In some embodiments, solution D may be stirred between at least 5,000 rpms and at most 10,000 rpms to facilitate mixing of solution A and solution B. In some embodiments, solution D may be stirred for at least 1 minutes to at most 24 hours. In some embodiments, solution D may be stirred for 5 minutes.

In step 512, solution C may be added to solution D to obtain solution E. In some embodiments, to facilitate mixing of solution C and solution D, solution E may be stirred between at least 5,000 rpms and at most 10,000 rpms, or between at least 7,500 rpms and at most 10,000 rpms, or between at least 7,500 rpms and at most 9,000 rpms, or between at least 7,500 rpms and at most 8,800 rpms. In some embodiments, solution D may be stirred for at least 1 minute to at most 24 hours. In some embodiments, the stir rate may be adjusted to prepare microcapsules of different sizes, shapes, or compositions. In some embodiments, solution E may be stirred at 8,800 rpm for 10 minutes, then 8,000 rpm for 10 minutes, then 7,500 for 10 minutes.

In step 514, solution E may be covered with an appropriate covering to prevent premature exposure to light. In some embodiments, solution E may be stirred in the dark or in a dark area to prevent premature light exposure. In some embodiments, solution E may be covered (e.g., with a metal covering or aluminum foil) to prevent premature exposure to light.

In step 516, solution E may be mixed, shook, or stirred at room temperature for a predetermined time to obtain a plurality of microcapsules of one or more of the embodiments disclosed herein dispersed in solution E. In some embodiments, solution E may be mixed on a shaker table at 180 rpm for 24 hours. In some embodiments, the plurality of microcapsules may have encapsulated portions of the payload. Optionally, in step 518, the microcapsules dispersed in solution E may be extracted to obtain the microcapsules in a solid form.

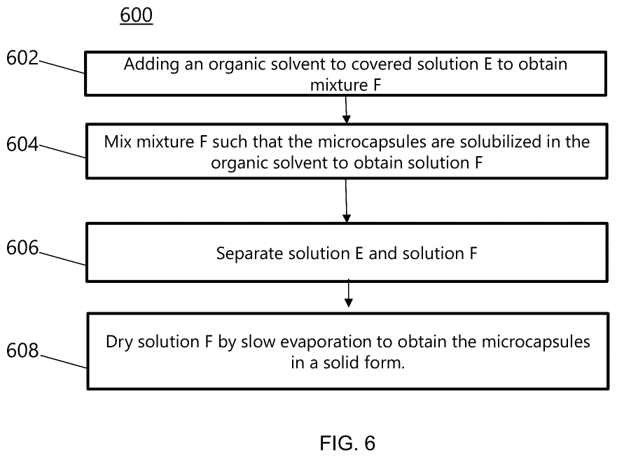

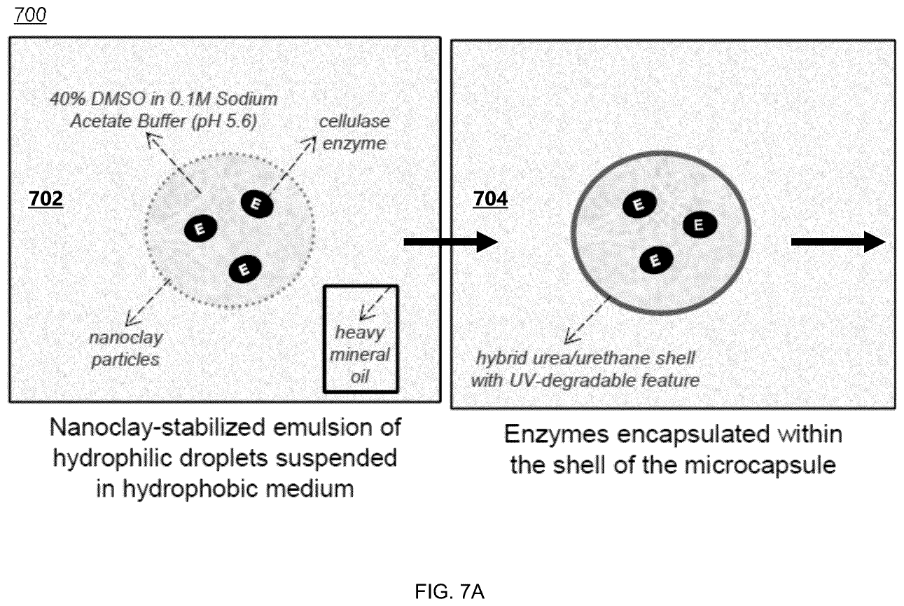

FIG. 6 depicts an example flowchart 600 for extracting example microcapsules according to one or more embodiments described herein to obtain the microcapsules in a solid form. The flowchart 600 may be implemented for any suitable microcapsules or delivery system. While an order of operations may be indicated in FIG. 6 for illustrative purposes, the timing and ordering of such operations may vary where appropriate without negating the purpose and advantages of the examples set forth in detail herein.

In step 602, after step 516 of FIG. 5, the microcapsules may be extracted from solution E by adding an organic solvent to the covered solution E to obtain mixture F. In some embodiments, the organic solvent may be an ethereal or a halogenated solvent. In some embodiments, the organic solvent may be diethyl ether. In some embodiments, mixture F may be a biphasic mixture resulting from the addition of the organic solvent.

In step 604, mixture F may be sufficiently mixed such that they microcapsules are solubilized in the organic solvent to obtain solution F. In some embodiments, sufficient mixing may be performed by shaking, inverting, or stirring the mixture.

In step 606, solution E and solution F may be separated using a suitable separation technique. In some embodiments, the separation technique may be a liquid-liquid extraction.

In step 608, solution F may be dried to obtain the microcapsules in a solid form. In some embodiments, the microcapsules may be dried using slow evaporation or freeze drying. In some embodiments, the microcapsules may contain residual solvent or water. In some embodiments, the residual solvent may be less than 10% by weight, less than 5% by weight, or less than 1% by weight of the solid microcapsules.

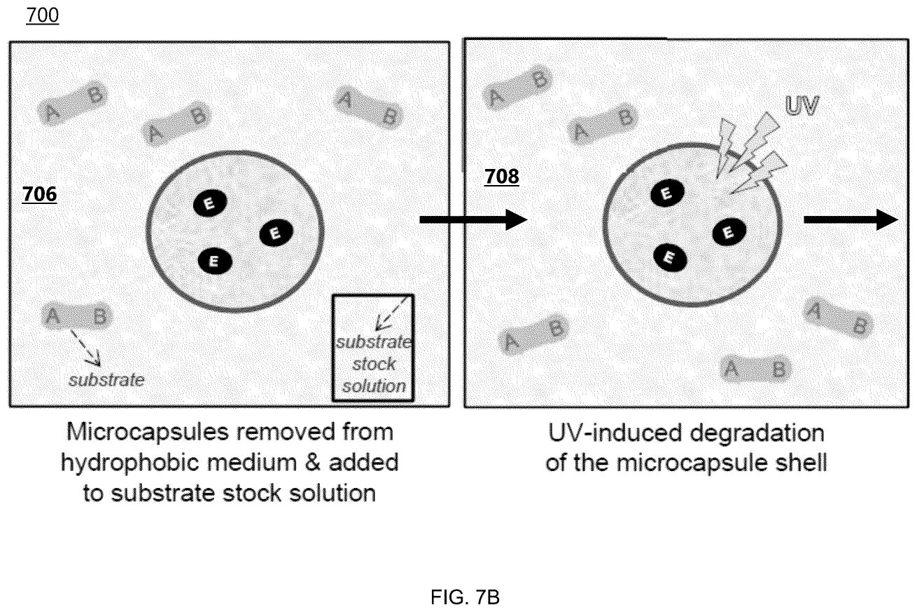

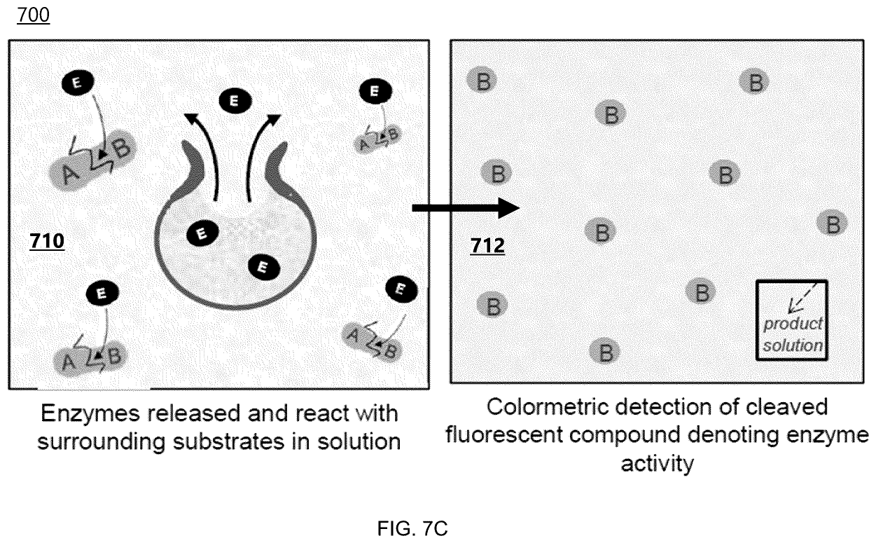

FIG. 7A, FIG. 7B, and FIG. 7C depict an example method 700 of delivering a payload to a target using example microcapsules according to one or more embodiments described herein. The example method 700 may be implemented for any suitable microcapsules or delivery system. FIG. 7A depicts example steps 702 and 704; FIG. 7B continues from FIG. 7A and depicts example steps 706 and 708; and FIG. 7C continues from FIG. 7B and depicts example steps 710 and 712. While an order of operations may be indicated in FIGS. 7A-7C for illustrative purposes, the timing and ordering of such operations may vary where appropriate without negating the purpose and advantages of the examples set forth in detail herein.

Step 702 depicts the formation of the microcapsule, where the hydrophilic droplets containing the payload (e.g., a cellulase enzyme) may be suspended in the hydrophobic medium. Step 704 depicts the formation of the microcapsules by encapsulating the payload within the oxime-based polymerization product according to one or more embodiments described herein. Step 706 depicts the microcapsules being applied to a target (e.g., a cellobioside-resorufin substrate solution). Step 708 depicts the UV induced degradation of the microcapsules. Step 710 depicts the release of the cellulase enzymes from the interior of the microcapsule and the binding of the cellobioside-resorufin substrate to the cellulase enzyme of the microcapsule. Step 712 depicts a colorimetric detection of the cleaved fluorescent compound (e.g., resorufin), which may indicate the cellulase enzyme was released from the microcapsule and maintained activity for the cellobioside-resorufin substrate throughout the process.

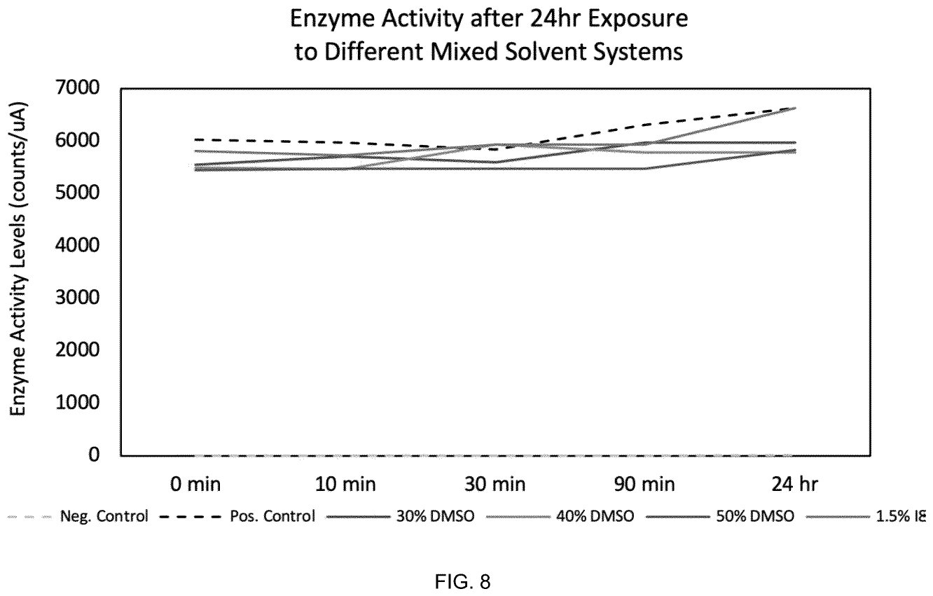

FIG. 8 depicts a chart of fluorimetry data from a control study of a cellulase enzyme, which may be used as the payload for example microcapsules discussed herein. According to the study, the enzyme activity may be unaffected by the addition of DMSO. This study was performed by first exposing a cellulase enzyme to various concentrations of DMSO in water and 1.5% sodium alginate-I8 in water for 24 hours. The cellulase enzyme was then removed and placed in a substrate stock solution that was covered in aluminum foil and stored in the refrigerator. As shown in FIG. 8, the enzyme maintained activity in all of the tested stock solutions.

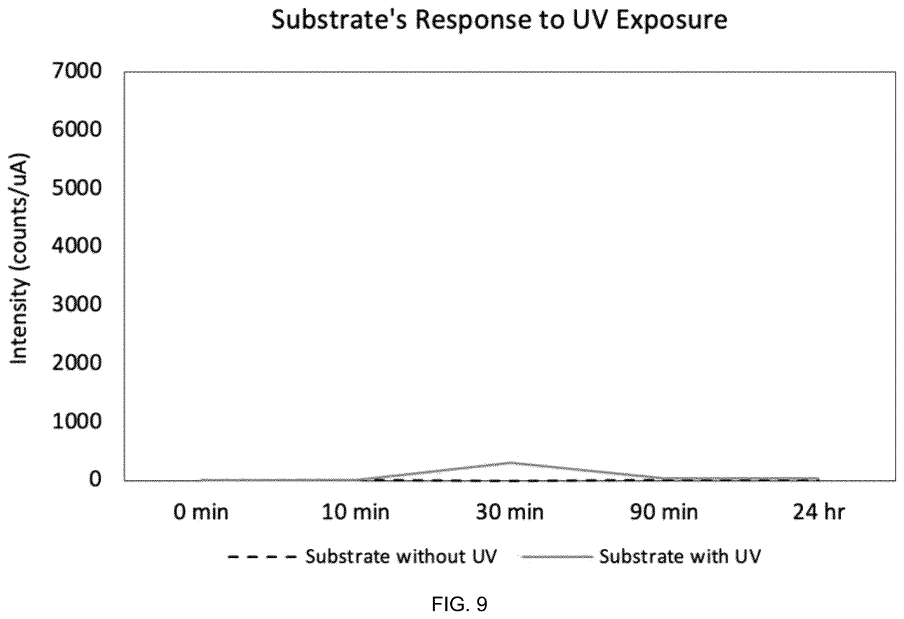

FIG. 9 depicts a chart of fluorimetry data from a control study of a cellobioside-resorufin substrate, which may be used as the target substrate for the cellulase enzyme payload, for example, microcapsules discussed herein. According to the study, the cellobioside-resorufin substrate may be unaffected by exposure to UV light. This study was performed by comparing two samples. The first sample contained a 0.5 mM solution of the cellobioside-resorufin substrate, which was not exposed to any UV light. The second sample also contained a 0.5 mM cellobioside-resorufin substrate, but was exposed to UV light with intermittent removal for testing after 10 minutes, 30 minutes, and 90 minutes. The sample was then left to sit for 24 hours with no UV exposure. The fluorescence of the resulting solution was then sampled at the 24 hour time point. As shown in FIG. 9, the fluorimetry data shows no change in intensity suggesting that the cellulase substrate may be unaffected by exposure to UV light.

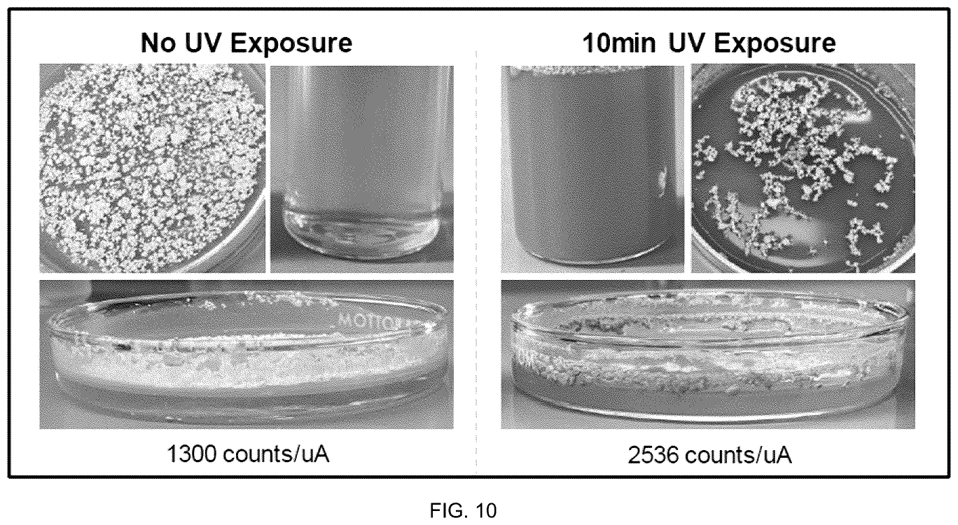

FIG. 10 depicts results from a comparative study between example microcapsules. In this study, example microcapsules that were kept in the dark were compared to example microcapsules that were exposed to UV light. To perform this study, two glass petri dishes containing 18 mL of 0.1 M sodium acetate buffer (pH 5.6) and 90 μL of 20% sodium dodecyl sulfate (SDS) to serve as the reaction medium were prepared. Next, 100 mg of dried microcapsule powder was sprinkled into each petri dish, and the contents were gently swirled to achieve an evenly dispersed layer of microcapsules across the top of the petri dish. For the petri dish being kept in the dark, 450 μL of the reaction buffer and 50 μL of the 5 mM cellobioside-resorufin substrate reagent were added to the mixture. The petri dish was then covered with aluminum foil to eliminate light exposure and the reaction was allowed to sit for 24 hours. The next day, the aluminum foil was removed, the reaction mixture was imaged, a 3 mL aliquot was taken from the petri dish and filtered through a 0.45 um syringe tip, and fluorescent measurements were taken at Ex=571 nm and Em=580 nm-620 nm using an Emission Scan type. For the petri dish being exposed to UV light, the petri dish was placed in the UV oven for 10 minutes at 100% intensity then removed and allowed to cool to room temperature. Then, 450 μL of the reaction buffer and 50 μL of the 5 mM cellobioside-resorufin substrate reagent were added to the mixture and the reaction was allowed to sit for 24 hours. The next day, the reaction mixture was imaged, a 3 mL aliquot was taken from the petri dish and filtered through a 0.45 um syringe tip, and fluorescent measurements were taken at Ex=571 nm and Em=580 nm-620 nm using an Emission Scan type. As shown in FIG. 10, the petri dish that was exposed to UV light (right side of FIG. 10) demonstrated almost double the fluorescence of the petri dish that was kept in the dark (left side of FIG. 10) indicating that the microcapsules had degraded, the cellulase enzyme had been released, and the cellulase enzyme reacted with the cellobioside-resorufin substrate to release the resorufin.

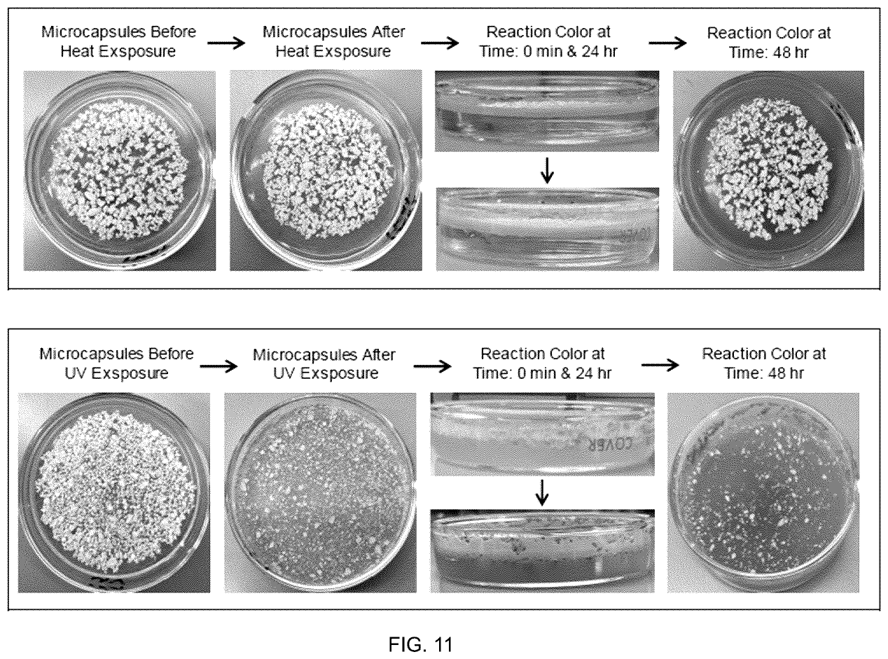

FIG. 11. depicts results from a comparative study between example microcapsules. In this study, example microcapsules that were exposed to heat were compared to example microcapsules that were exposed to UV light. To perform this study, two glass petri dishes containing 18 mL of 0.1 M Sodium Acetate buffer (pH 5.6) and 90 μL of 20% SDS to serve as the reaction medium were prepared. Next, 100 mg of dried microcapsule powder was sprinkled into both petri dishes, and the contents were gently swirled to achieve an evenly dispersed layer of microcapsules across the top of the petri dish. One of the petri dishes was then placed in a thermal oven for 10 minutes at 50° C., then removed and allowed to cool to room temperature, while the other petri dish was placed in a UV oven for 10 minutes at 100% intensity then removed and allowed to cool to room temperature. Afterwards, 450 μL of reaction buffer and 50 μL of 5 mM cellobioside-resorufin substrate reagent were added to both petri dishes, and each reaction was allowed to sit for 24 hours. As shown in the top of FIG. 11, the petri dish that was exposed to heat showed minimal color change and minimal degradation of the microcapsules indicating that the microcapsules of the invention are stable to heat. As shown in the bottom of FIG. 11, the petri dish that was exposed to UV light demonstrated substantial degradation of the microcapsules and underwent a substantial color change indicating the cellulase enzyme had been released and reacted with the cellobioside-resorufin substrate to release the resorufin.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention need not be limited to the described embodiments, but that it has the full scope defined by the language of the following claims, and equivalents thereof.

What is claimed is:

1. A microcapsule comprising an oxime-based polymerization product of two or more monomers, wherein a payload is encapsulated within the microcapsule.

2. The microcapsule of claim 1, wherein the oxime-based polymerization product is comprised of an oxime-based monomer and an isocyanate-based monomer.

3. The microcapsule of claim 2, wherein the oxime-based monomer contains one or more oxime functional groups.

4. The microcapsule of claim 2, wherein the oxime-based monomer is selected from the group consisting of:

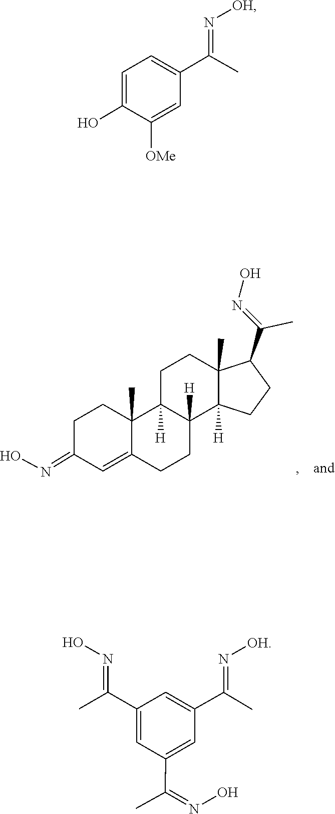

5. The microcapsule of claim 2, wherein the isocyanate-based monomer contains two or more isocyanate functional groups.

6. The microcapsule of claim 2, wherein the isocyanate-based monomer is selected from the group consisting of isophorone diisocyanate, methylene diisocyanate, hexamethylene diisocyanate, and toluene diisocyanate.

7. The microcapsule of claim 2, wherein the oxime-based polymerization product further comprises a polymeric amine.

8. The microcapsule of claim 7, wherein the polymeric amine is selected from the group consisting of polyethyleneimine, (poly)allylamine, polyvinylamine, poly(4-aminostyrene), and chitosan.

9. The microcapsule of claim 7, wherein the oxime-based monomer, isocyanate-based monomer, and the polymeric amine are in a 1:2:1 ratio.

10. The microcapsule of claim 1, wherein the microcapsule is a solid powder or dispersed in a solution.

11. The microcapsule of claim 1, further comprising an interior, wherein the interior of the microcapsule comprises the payload and an aqueous solution.

12. The microcapsule of claim 1, wherein the payload is water-soluble.

13. The microcapsule of claim 1, wherein the payload is an enzyme, a catalyst, or a pharmaceutical.

14. The microcapsule of claim 1, wherein the microcapsule degrades after exposure to ultraviolet light.

15. The microcapsule of claim 1, wherein the microcapsule is at least 1 micron to at most 1000 microns in diameter.

16. A method of preparing a plurality of microcapsules, the method comprising:
preparing a solution A comprising a nanoclay and mineral oil;
preparing a solution B comprising a payload and an oxime-based monomer;
preparing a solution C comprising an isocyanate-based monomer and mineral oil;
adding a polymeric amine to solution B;
adding solution B to solution A to obtain a solution D;
adding solution C to the solution D to obtain a solution E;
covering solution E to prevent exposure to light; and
at least one of mixing, shaking, or stirring the covered solution E at room temperature for a predetermined time to obtain the plurality of microcapsules dispersed in solution, the microcapsules encapsulating portions of the payload.

17. The method of claim 16, further comprising extracting the microcapsules dispersed in solution to obtain the microcapsules in solid form, wherein extracting the microcapsules comprises:
adding an organic solvent to the covered solution E to obtain a mixture F;
mixing mixture F such that the microcapsules are solubilized in the organic solvent to obtain solution F;
separating solution E and solution F; and
drying solution F by slow evaporation to obtain the microcapsules in solid form.

18. The method of claim 16, wherein solution B further comprises a polar aprotic solvent.

19. The method of claim 16, wherein at least one of mixing, shaking, or stirring the covered solution E comprises stirring the covered solution E between at least 5,000 rpm to at most 10,000 rpm.

20. A method of delivering a payload to a target, the method comprising:
applying microcapsules to the target, the microcapsules encapsulating the payload, the microcapsules comprising an oxime-based polymerization product of two or more monomers; and
exposing the target to ultraviolet light to decompose the microcapsule and release the payload to the target.
